(19) United States
(12) Patent Application Publication
(10) Pub. No.: US 2025/0082671 A1
(43) Pub. Date: Mar. 13, 2025

(54) ANIMAL FEED ADDITIVE COMPOSITION, PRE-MIX FEED COMPOSITION AND MANUFACTURING METHOD FOR SUB CLINICAL HYPCALCEMIA RELIEF, ENERGY IMPROVEMENT AND INCREASED MILK PRODUCTION

(71) Applicant: Chemlock Metals Corp., Cincinnati, OH (US)

(72) Inventors: Arun Mehan, Cincinnati, OH (US); Grant T. Shouldice, Cincinnati, OH (US)

(73) Assignee: Chemlock Metals Corp., Cincinnati, OH (US)

(21) Appl. No.: 18/826,701

(22) Filed: Sep. 6, 2024

Related U.S. Application Data
(60) Provisional application No. 63/537,289, filed on Sep. 8, 2023.

(51) Int. Cl. A61K 33/06; A23K 20/28; A23K 40/20; A23K 50/10; A61K 9/16; A61K 33/24; A61P 3/14

(52) U.S. Cl. CPC A61K 33/06; A23K 20/28; A23K 40/20; A23K 50/10; A61K 9/1611; A61K 33/24; A61P 3/14

(57) ABSTRACT

A composition is provided that includes a synthetic sodium aluminosilicate structure having the Formula: Na₁₂[(AlO₂)₁₂(SiO₂)₁₂·27H₂O (I). A chelate of the Formula (I) or (II) intercalated, associated, or co-administered with the synthetic sodium aluminosilicate:

[R(O)O⁻]ₘ . . . Cr(³⁺)X⁻(H₂O)ₙ (I)

or _{Z}[R((O)O)⁻)₂]ₘ . . . Cr(³⁺)X⁻(H₂O)ₙ (II)

where R is a C₃-C₈ aliphatic, C₆-C₁₄ aromatic, or a substituted C₃-C₈ aliphatic or C₆-C₁₄ aromatic in which a proton is replaced with an amino, thio, hydroxyl, or C₂-C₄ aliphatic with the proviso that a nitrogen substitution can occur within an aromatic ring and still maintaining aromaticity; X is a counterion of a halide, polyatomic anion present at a stoichiometric ratio relative to Cr(³⁺) to form a neutral compound; n is an integer number of 0-6 to satisfy the coordination number of Cr(³⁺); and m is an integer value of 2 or 3. The composition is useful in a method of preventing or treating a ruminant for hypocalcemia.

### RELATED APPLICATIONS

This application claims priority benefit of U.S. Provisional Application Ser. No. 63/537,289 filed Sep. 8, 2023; the contents of which are hereby incorporated by reference.

### FIELD OF THE INVENTION

The present invention provides compositions of a feed additive and method of manufacturing thereof, for preventing parturient sub clinical hypocalcemia in animals and in particular there is provided a feed additive for increasing ruminate milk production.

### BACKGROUND OF THE INVENTION

Calcium (Ca²⁺) is one of the main cations and metabolic actors for dairy cows and it is involved in several key biological functions such as muscle contraction, muscle signaling, neurotransmission, intracellular signaling and bone metabolism. Hypocalcemia, which is the nutritional metabolic disorder caused by inadequate levels of calcium ions, remains one of the primary disorders that disrupts production on dairy farms and results in major economic losses around the world. It is generally known that hypocalcemia is not related to inadequate intake of calcium from dietary sources rather, it is caused by the inability of the cow to mobilize calcium from bones fast enough at the onset of colostrogenesis and lactation. The clinical phase of hypocalcemia is defined by the content of calcium in the plasma of the cow lower than ‘serum Ca’ less than 6.0 mg/dl or 1.5 mmol/L. The less overt subclinical hypocalcemia, although symptomatically similar to clinical hypocalcemia, is characterized by a reduced blood calcium concentration to between 6.0 mg/dl but lower than 8.8 mg/dl or 1.5 to 2.2 mmol/L.

Generally, hypocalcemia affects 2%-10% of a herd and is characterized by clinical symptoms including but not limited to, cold extremities, lateral decubitus, apathy, and staking. It can directly cause the reduction of the feed intake of the cow, the peristalsis of the rumen and the small intestine, cause the risk of abomasum displacement and reduction of the milk yield. Furthermore, it can then lead to the development of other diseases including but not limited to, mastitis, metritis, endometritis, displacement of the abomasum and even death. Moreover, it is generally known that as a direct result of the subclinical version of the disorder, which can affect up to 50% of a herd, the energy level of the animal is reduced such that normal activity is characterized by lethargy and apathy in, including but not limited to, movement, eating, and milk production. Generally, there is a reduction in milk production of about 3.0 L/day of milk due to sub clinical hypocalcemia. During the transition period from a pregnant/non-lactating to lactating/non-pregnant state that includes three weeks of peripartum, and three weeks of postpartum, the animal undergoes several physiological changes. The demand for calcium increases significantly during the peripartum period because of the final formation of the calf and colostrum production. The background demand for calcium is approximately 30 g/day while in the postpartum period during milk production it can reach up to 100 g/day. Thus, this is a critical period for maintaining health and productivity of the animal.

One of the most widely used methods to protect dairy cows against hypocalcemia is to modify the mineral composition of their prepartum diet. Anionic salts are added to cause a mild acidosis in the animal, which can activate homeostatic mechanisms thereby causing greater calcium mobilization of the gastrointestinal tract, renal reabsorption and bone mobilization. This is based on the premise that cows in metabolic acidosis are more sensitive to parathyroid hormone (PTH) and that giving acidogenic salts can increase the expression of renal PTH receptors. The maximum PTH response to calcium chelation can be leveraged when the blood pH of a cow is in a metabolic acidosis state. Thus, peripartum diets have been altered to induce a compensated metabolic acidosis state in the last few weeks of gestation to reduce the risk of hypocalcemia. However, this diet has met with limited acceptance owing to the high cost of salts and the reduction in dry matter intake due to the acidosis. In addition, improper dosing can cause severe acidosis, resulting in various other metabolic dysfunctions. It is also necessary to treat the animal's diet for several days to have a satisfactory effect. Regardless of the careful usage, a high incidence of hypocalcemia is still observed when employing this type of strategy.

Another, less common, strategy to address hypocalcemia is to administer intravenous (IV) calcium shortly after delivery. This is capable of rapidly increasing plasma levels of calcium. However, intravenous calcium is soon excreted, and the circulating concentration thereafter typically falls below the physiological level prior to IV administration. A generally accepted hypothesis for this observation is that the IV calcium does not activate the homeostasis mechanisms thereby leaving the animal still susceptible to hypocalcemia.

Still another strategy to address hypocalcemia is to administer oral calcium supplements, either in the form of a solution or a bolus. Both intravenous and bolus strategies are based on increasing the availability of calcium for the animal. Generally, the circulating calcium concentration remains low after administration thereby leaving the animal still susceptible to hypocalcemia.

Still another approach of preventing hypocalcemia and/or subclinical hypocalcemia is the peroral drenching with calcium chloride around calving, as detailed in U.S. Pat. No. 6,890,550. The effect of this method is interpreted as a simple calcium diffusion through the walls of the forestomach and the small intestine. This method has met with limited acceptance owing to the complexity that the dairy cows have to be handled and dosed individually. Furthermore, the precise calving time of the cow must be known since the first drenching is recommended to be carried out a few hours before calving.

Injection of a bioactive synthetic D-Vitamin preparation in large doses has been suggested for the prevention and/or treatment of hypocalcemia and/or subclinical hypocalcemia. Although this method is documented to be effective, it is not allowed in many countries, and is both expensive and labor intensive.

Still another suggestion involves continuous milking, which will prevent hypocalcemia and/or subclinical hypocalcemia, as the calcium regulating mechanisms are constantly activated. However, this method is only applicable to single cows which are known or suspected to present hypocalcemia. As a result, continuous milking is not feasible at a herd level and requires considerable amounts of testing.

It has been observed that hypocalcemia can be controlled and/or eliminated by administering a low calcium dietary ration to dairy cows during the weeks immediately prior to calving. Ramberg et al., “Calcium kinetics in cows during late pregnancy, parturition, and early lactation;” Am. J. of Physiology, (1970) 219:5, 1166-1177. By feeding a low calcium dietary ration, the cow is left in a negative calcium balance, which results in the activation of its natural calcium regulating mechanisms. Unfortunately, there are many limitations associated with this method. For example, these limitations include that most of the cattle feed, such as alfalfa and other legumes, naturally contain calcium levels that are too high to bring the dry cow into a negative calcium balance. Furthermore, the method presents a substantial problem when dealing with a large herd. To solve the former problem, U.S. Pat. No. 3,908,020 discloses a complete, calcium-limited feed ration. This method has met with limited acceptance as the purchase of rather expensive feed is not feasible under commercial dairy farming conditions where the farmer is using home-grown crops for feeding.

In still another strategy to address hypocalcemia, a combination of sodium aluminosilicates (bioactive synthetic or natural occurring) with various forms of zinc salts and/or chelating agents either added to the cow's drinking water or administered perorally such that the calcium ions are absorbed from the drinking water or the cow's ration are reduced. M. A. Crookenden et al. “Feeding Bioactive synthetic zeolite to transition dairy cows alters neutrophil gene expression;” J. of Dairy Sci. (2020), 103(1), 723-736.

One of the main observations in the field is lethargic and/or apathetic behavior of both peripartum and postpartum cows. In fact, during the early stages of bovine lactation, defined as less than 100 days postpartum, and especially in high yield cows, negative energy balance, breeding difficulty and postpartum disease incidence increases even in cows whose calcium levels remain outside the subclinical definition of hypocalcemia.

Chromium is primarily known for its role in glucose metabolism and insulin regulation, rather than its direct involvement in calcium metabolism. However, negative energy balance not only influences milk production but also influences the cow reproduction performance and increases the incidence of sub-clinical hypocalcemia and other metabolic disorders. Essential Chromium (Cr) is known to improve dry matter intake (DMI) and therefore increase energy and milk production. In fact, Cr supplementation is known to increase DMI, however, the response to supplementation is less before parturition than after parturition. For multiparous cows compared to the un-supplemented ones, the DMI is on average higher with Cr supplementation in the form of chromium propionate complex. Furthermore, with Cr supplementation milk production was observed to increase by an average of 1.2 kg. For multiparous and primiparous cows, milk production increased by an average of 1 kg and 2 kg, respectively. It should be readily apparent that a slippery slope exists with respect to sub-clinical hypocalcemia and clinical hypocalcemia that begins with a negative energy balance related to DMI and ends in clinical treatments for hypocalcemia.

Thus, there remains a need for new compositions for the prophylaxis or treatment of hypocalcemia. There also exists a need for new methods for addressing hypocalcemia. There further exists a need for a herd wide prophylactic method of precluding sub-clinical hypocalcemia and clinical hypocalcemia. There also exists a need for a treatment method that does not require laboratory testing.

### SUMMARY OF THE INVENTION

A composition is provided that includes a synthetic sodium aluminosilicate structure having the Formula: Na₁₂[(AlO₂)₁₂(SiO₂)₁₂·27H₂O (I). A chelate of the Formula (I) or (II) intercalated, associated, or co-administered with the synthetic sodium aluminosilicate:

[R(O)O⁻]ₘ . . . Cr(³⁺)X⁻(H₂O)ₙ (I)

or _{Z}[R((O)O)⁻)₂]ₘ . . . Cr(³⁺)X⁻(H₂O)ₙ (II)

where R is a C₃-C₈ aliphatic, C₆-C₁₄ aromatic, or a substituted C₃-C₈ aliphatic or C₆-C₁₄ aromatic in which a proton is replaced with an amino, thio, hydroxyl, or C₂-C₄ aliphatic with the proviso that a nitrogen substitution can occur within an aromatic ring and still maintaining aromaticity; X is a counterion of a halide, polyatomic anion present at a stoichiometric ratio relative to Cr(³⁺) to form a neutral compound; n is an integer number of 0-6 to satisfy the coordination number of Cr(³⁺); and m is an integer value of 2 or 3.

The composition is useful in a feed additive or feed composition. A method of preventing or treating a ruminant for hypocalcemia therewith is also provided.

### DETAILED DESCRIPTION OF THE INVENTION

The present invention has utility as a food additive composition, pre-mix feed composition and manufacturing method for sub-clinical hypocalcemia relief, energy improvement and increased milk production. A bioactive synthetic sodium aluminosilicate molecule in combination with a chelated metal ion is provided. In some inventive embodiments, an inventive composition is effective in the prophylaxis of sub clinical hypocalcemia symptoms. Synergistic effects are noted with bioactive synthetic sodium aluminosilicate molecule combined with micronutrient formulations of chromium.

In other inventive embodiments, a method of manufacturing the inventive composition is provided. In still other inventive embodiments, the composition is effective for preventing parturient sub-clinical hypocalcemia in gestating animals. The composition is amenable to addition to animal feed mix or premix during the dry period for the prevention of sub-clinical hypocalcemia and/or milk fever while improving energy levels and milk production of a lactating ruminant.

The present invention will now be described with reference to the following embodiments. As is apparent by these descriptions, this invention can be embodied in different forms and should not be construed as limited to the embodiments set forth herein. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references mentioned herein are incorporated by reference in their entirety.

It is to be understood that in instances where a range of values are provided that the range is intended to encompass not only the end point values of the range but also intermediate values of the range as explicitly being included within the range and varying by the last significant figure of the range. By way of example, a recited range from 1 to 4 is intended to include 1-2, 1-3, 2-4, 3-4, and 1-4.

### Definitions

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also, as used herein, “and/or” refers to and encompasses any and all possible combinations of one or more of the associated listed items, as well as the lack of combinations when interpreted in the alternative (“or”).

As used herein, the term “ruminant” refers to domesticated cattle, goats, and sheep.

An inventive composition includes a bioactive synthetic sodium aluminosilicate structure, having the general formula Na₁₂[(AlO₂)₁₂(SiO₂)₁₂·27H₂O. The bioactive synthetic sodium aluminosilicate structured molecule has a sodium content is 5-16% and aluminum content is from 5-19% of the total by weight. Other inventive embodiments, the sodium content is 8-13% and aluminum content is from 9-15% of the total by weight.

The bioactive synthetic sodium aluminosilicate structured molecule is combined with chelate of the Formula (I) or (II):

[R(O)O⁻]ₘ . . . Cr(³⁺)X⁻(H₂O)ₙ (I)

or [R((O)O)⁻)₂]ₘ . . . Cr(³⁺)X⁻(H₂O)ₙ (II)

where R is a C₃-C₈ aliphatic, C₆-C₁₄ aromatic, or a substituted C₃-C₈ aliphatic or C₆-C₁₄ aromatic in which a proton is replaced with an amino, thio, hydroxyl, or C₂-C₄ aliphatic with the proviso that a nitrogen substitution can occur within an aromatic ring and still maintaining aromaticity; X is a counterion of a halide, polyatomic anion present at a stoichiometric ratio relative to Cr(³⁺) to form a neutral compound; n is an integer number of 0-6 to satisfy the coordination number of Cr(³⁺); and m is an integer value of 2 or 3. Specific examples of R—(O)OH and R—((O)(OH)₂ as the free acids prior to forming the chelate illustratively include the monoacids of acetic acid, glycolic acid, glutaric acid, butyric acid, propionic acid, lactic acid, glyceric acid, pyruvic acid, malonic acid, valeric acid, aminovaleric acid, glycinic acid and picolinic acid; and diacids of succinic acid, itaconic acid (2-methylenesuccinic acid), adipic acid, 2,5-furandicarboxylic acid.

In some inventive embodiments, the composition is simply bioactive synthetic sodium aluminosilicate into which a chelate of Formula (I) or (II) resides on the surface of the bioactive sodium aluminosilicate. Without intending to be bound to a particular theory, bioactive sodium aluminosilicate molecule is believed to exchange sodium for gastrointestinal tract calcium thereby bringing calcium into proximity to the metal sites of the chelate to increase circulatory levels of calcium to levels not obtainable with either inventive component alone.

The admixture of the bioactive synthetic sodium aluminosilicate with the compound of Formula (I) or (II) in some embodiments is combined with other components, such as carrier particle to form a storage stable pre-mix feed composition amenable to mixing with feed for any individual animal as well as a herd.

A method of forming an inventive admixture includes dispersing a bioactive synthetic sodium aluminosilicate structured molecule with ruminant ingestion compatible carrier particles to form a mixture. The mixture is then processed in several synthetic steps, extruded, and then treated with a solution of Formula (I), Formula (II), or a combination thereof to form the admixture. Typical weight ratios of the mixture to the isolated compounds of Formulas (I) in solution or (II) in range from 0.02-0.2:1. Typical weight ratios of the mixture to the isolated compounds of Formulas (I), powder form, or (II) range from 0.2-2:1. In the powder form, the isolated compounds of Formulas (I) or (II) are from 0.1 to 0.5% in a pellet. In some inventive embodiments, the bioactive synthetic sodium aluminosilicate structure is present from 70-95% by weight (of the admixture); the carrier particles from 5-30% by weight; and the chelate being the remainder that is often from 0.1-20%.

In some inventive embodiments, the chelate metal is chromium. In still other embodiments the chromium is chelated by propionate. Without intending to be bound to a particular theory, an inventive admixture containing chromium chelates is observed to increase calcium levels in vivo in ruminates thereby alleviating the sub-clinical hypocalcemia symptoms while also improving energy and therefore milk production in a synergistic manner suggesting that the chromium so delivered is metabolically available and working concomitantly.

Carrier particles operative herein illustratively include amylose, amylopectin, corn starch, potato starch, tapioca starch, rice starch, wheat starch, arrowroot starch, cassava starch, sweet potato starch, barley starch, oat starch, rye starch, sorghum starch, millet starch, glucose, fructose, galactose, sucrose, lactose, maltose, dextrose, ribose, xylose, mannose, arabinose, trehalose, sorbital, maltitol, lignosulfonates, maltodextrin, cyclodextrin, amylodextrin, achrodextrin, limit dextrin, white dextrin and British gum 1-7.

Other adjuvants that can be present in the carrier particles illustratively include at least one of calcium, phosphorus, magnesium, potassium, sodium, vitamin A, vitamin D, vitamin E, fats and oils like soybean oil or vegetable oil, probiotics, yeast, enzymes, or rumen buffers, and other anti-oxidants, grass hay, or silage. The amount of a given adjuvant in the carrier particles typically range between 0.05 and 5 percent by weight of the carrier particle, if present.

In still other inventive embodiments, the compound of Formula (I) or (II) is combined with a carrier particle and detailed above or in an inorganic matrix such as a calcium salt. The chelate dosed carrier particles is amenable to being mixed with bioactive synthetic sodium aluminosilicate dosed carrier particles to form a two-part feed supplement. The two-part feed supplement is also storage stable as separate or combined components and is also amenable to being mixed with conventional ruminant feeds.

An inorganic powder is readily formed with calcium carbonate by techniques detailed in U.S. Pat. No. 6,613,138B2 with which a solution of inventive chelate is intermixed as a powder or solution. Typically loading of chromium in such a pellet are between 0.01-2% by pellet weight.

A doughy mixture of the bioactive synthetic sodium aluminosilicate and carrier particles is amenable to extrusion. Extruders operative herein include a single screw extruder, a twin screw extruder, a co-extruder, a hot melt extruder, a cold feed extruder, a ram extruder, a direct extruder, an indirect extruder, a continuous extruder or a batch extruder. Typical die sizes for extrusion are from 1/32- 1/16 inch in diameter. The extrudate is readily chopped or crumbled to form ingestible sized pellets. Typical pellet sizes are from 0.1 to 5 mm in length.

The composition of the mixture is detailed in Table 1.

TABLE 1
Composition of bioactive synthetic
aluminosilicate structured molecule

| Raw Material | Mass Fraction |
|---|---|
| Sodium Aluminate | 30-60 |
| Sodium Silicate | 30-60 |
| Sodium Hydroxide | 1-5 |
| Water | remainder |
| Carrier Particle | 5-15 |

The formation of a chelate of Formula (I) or (II) is known to the art. JP Collman et al. “Reactions of Metal Chelates. V.1,2 Substitution of Metal Acetylacetonates with Friedel-Crafts Acylating Reagents and Sulfur Electrophiles”; J. Org. Chem. 1963, 28, 6, 1449-145. The synthesis of a metal chelate of Formula (I) or (II) typically occurs under mild conditions of standard pressure and temperatures of less than 100° C. in aqueous solution, or conditions as detailed in the cited literature. The ratios of components for formation of a chelate solution operative in the present invention are detailed in Table 2.

TABLE 2
Composition of Solution containing Formula (I) or (II)

| Raw Material | Mass Fraction |
|---|---|
| Metal Chloride | 5-30 |
| Water | remainder |
| Propionic Acid | 2-3 ± 10% of molar amount of metal chloride |

It is appreciated that other water-soluble metal salts are operative in the formation of an inventive chelate. These salt anions illustratively include nitrates, perchlorates, sulfates, sulfonates, bromides, iodates, phosphates, phosphonates, hydrides of, and combinations of any of the aforementioned. To the extent that a given anion is undesirable in a feed, the resulting compound of Formula (I) or (II) is readily dialyzed or treated with an ion exchange resin. The compound of Formula (I) or (II) typically is present from 0.1 to 20 weight percent of an aqueous solution used for application to the bioactive synthetic sodium aluminosilicate component, alone or in combination with carrier particles.

An inventive composition or admixture thereof is storage stable, and able to be mixed with any conventional ruminant feed. Typical dosing found to be effective prophylaxis of sub clinical hypocalcemia symptoms are from 0.001% to 0.005% by weight of the feed of the Cr(³⁺).

The present invention is further detailed with respect to the following non limiting examples. These examples are not intended to limit the scope of the invention but rather highlight properties of specific inventive embodiments and the superior performance thereof relative to comparative examples.

### EXAMPLES

### Example 1. Method, Order of Addition, Conditions, Equipment for Chromium Propionate Single Component Composition

### Steps:

1. Prepare a solution of sodium aluminate (NaAlO₂) and sodium silicate (Na₂SiO₃) in water. The molar ratio of sodium aluminate to sodium silicate should be 1:1±30%.
2. Mix the solution thoroughly to ensure the sodium aluminate and sodium silicate are well dissolved.
3. Adjust the pH of the solution to 9-12 using sodium hydroxide (NaOH) or other suitable base source such a ammonia or potassium hydroxide.
4. Transfer the solution to a high-pressure autoclave or reactor vessel. The vessel should be able to withstand high temperatures and pressures.
5. Seal the vessel and heat it to a temperature between 6° and 150° C.
6. Maintain the temperature and pressure inside the vessel for a specific duration, typically ranging from 12 to 48 hours during which time the bioactive synthetic sodium aluminosilicate crystals form and grow.
7. Add carrier particles to the crystal slurry and begin a slow kneading process.
8. Cool down the vessel slowly to room temperature while kneading the material.
9. Collect the kneaded “doughy” like material and send to an extruder outfitted with a die opening, Typical die sizes are on the order of 1/32 to 1/16 inch in diameter.
10. The bioactive synthetic aluminosilicate and carrier particle mixture is extruded and dried.
11. The extrudate is amenable to be chopped or crumbled into pellets of from 0.1-5 mm.
12. Move the pellets to an auger mixer bed equipped with a spray tank and nozzle. Spray the aqueous chromium propionate solution onto the pellets until a homogenous admixture results. The admixture being storage stable under dry barn conditions for more than 36 months.

### Example 2. Preparation of Chromium Propionate Solution

### Steps:

1. Dissolve chromium chloride in water to form a 20% by weight solution.
2. Propionic acid is added to the chromium chloride solution at twice the chromium stoichiometry.
3. The solution is heated to a temperature between 80-95° C. and maintained for 1-3 hours to yield a chelate solution.
4. The resulting solution is cooled, and solid impurities filtered therefrom before addition at Step 11 of Example 1.

### Example 3. Chromium Succinate

The process of Example 1 is repeated with the chromium propionate replaced by chromium succinate to achieve a like inventive admixture composition.

### Example 4. Cattle Feeding with Chromium Propionate

The admixture composition of Example 1 represents a ruminant feed supplement that is mixed into dairy cattle feed at a total content of the chromium, from 0.001% to 0.005% and feed to an entire herd or single animal on a daily basis for up to 21 days. Thereafter, sub clinical hypocalcemia symptoms are observed to decrease significantly and those animals previously suffering from sub clinical hypocalcemia symptoms are adding weight and milk production, indicating reversal of the hypocalcemia without resort to blood testing. Overall milk production is observed to increase relative to comparable negative control herd not feed with the feed supplement admixture of Example 1.

### Example 5. Cattle Feeding with Chromium Succinate

The process of Example 4 is repeated with the composition of Example 3 with similar results.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient road map for implementing the exemplary embodiment or exemplary embodiments. It should be understood that various changes may be made in the function and arrangement of elements without departing from the scope as set forth in the appended claims and the legal equivalents thereof.

The foregoing description is illustrative of particular embodiments of the invention, but is not meant to be a limitation upon the practice thereof. The following claims, including all equivalents thereof, are intended to define the scope of the invention.

1. A composition comprising:
a synthetic sodium aluminosilicate structure having the Formula: Na₁₂[(AlO₂)₁₂(SiO₂)₁₂·27H₂O; and
a chelate of the Formula (I) or (II) intercalated, associated, or co-administered with the synthetic sodium aluminosilicate:
[R(O)O⁻]ₘ . . . Cr(³⁺)X⁻(H₂O)ₙ (I)
or _{Z}[R((O)O)⁻)₂]ₘ . . . Cr(³⁺)X⁻(H₂O)ₙ (II)
where R is a C₃-C₈ aliphatic, C₆-C₁₄ aromatic, or a substituted C₃-C₈ aliphatic or C₆-C₁₄ aromatic in which a proton is replaced with an amino, thio, hydroxyl, or C₂-C₄ aliphatic with the proviso that a nitrogen substitution can occur within an aromatic ring and still maintaining aromaticity; X is a counterion of a halide, polyatomic anion present at a stoichiometric ratio relative to Cr(³⁺) to form a neutral compound; n is an integer number of 0-6 to satisfy the coordination number of Cr(³⁺); and m is an integer value of 2 or 3.

2. The composition of claim 1 wherein R(O)O⁻ is propionate.

3. The composition of claim 1 wherein R is a portion of a monoacid.

4. The composition of claim 3 wherein the monoacid is independently in each occurrence acetic acid, glycolic acid, glutaric acid, butyric acid, propionic acid, lactic acid, glyceric acid, pyruvic acid, malonic acid, valeric acid, aminovaleric acid, glycinic acid, picolinic acid.

5. The composition of claim 1 wherein R is a portion of a diacid.

6. The composition of claim 5 wherein the diacid is independently in each occurrence of succinic acid, itaconic acid (2-methylenesuccinic acid), adipic acid, 2,5-furandicarboxylic acid.

7. The composition of claim 1 wherein the synthetic sodium aluminosilicate structured molecule has a sodium content is 5-16% and aluminum content is from 5-19% of the total by weight.

8. A feed additive or feed composition comprising:
a composition of claim 1; and
carrier particles mixed with at least one of the synthetic sodium aluminosilicate structure or the chelate of the Formula (I) or (II) are formed into pellets, and optionally a bulk ruminant feed in which the composition of claim 1 and the carrier particles are intermixed.

9. The feed additive or feed composition of claim 8 wherein the carrier particles includes at least one of: amylose, amylopectin, corn starch, potato starch, tapioca starch, rice starch, wheat starch, arrowroot starch, cassava starch, sweet potato starch, barley starch, oat starch, rye starch, sorghum starch, millet starch, glucose, fructose, galactose, sucrose, lactose, maltose, dextrose, ribose, xylose, mannose, arabinose, trehalose, sorbital, maltitol, lignosulfonates, maltodextrin, cyclodextrin, amylodextrin, achrodextrin, limit dextrin, white dextrin or British gum 1-7.

10. The feed additive feed additive or feed composition of claim 8 wherein the bioactive synthetic sodium aluminosilicate structure is present from 70-95% by weight of the admixture.

11. The feed additive or feed composition of claim 8 wherein the synthetic sodium aluminosilicate structure or the chelate of the Formula (I) or (II) are present within a single pellet or are each present in a separate type of pellet.

12. The feed additive or feed composition of claim 8 wherein the chelate of the Formula (I) or (II) is present in an inorganic matrix.

13. The feed additive or feed composition of claim 12 wherein the inorganic matrix is calcium carbonate.

14. The feed additive or feed composition of claim 8 wherein the bulk ruminant feed is present and the composition and the carrier particles together are present from 1% to 2% by weight.

15. The feed additive or feed composition of claim 14 wherein mass fractions of Al, Na, and Cr are 0.05%-0.4% by weight, 0.05%-0.32% by weight, and 0.001%-0.01% by weight, respectively.

16. A method of preventing or treating a ruminant for hypocalcemia comprising:
administering to the ruminant the composition of claim 1; and
allowing sufficient time to prevent or treat the hypocalcemia.

17. The method of claim 16 wherein the administering is simultaneous to a herd including the ruminant.

18. The method of claim 16 further comprising not performing calcium level testing of the ruminant or the herd.

19. The method of claim 16 wherein the composition is administered mixed with bulk ruminant feed.

20. The method of claim 19 wherein the composition is combined with carrier particles.
